(19) United States
(12) Patent Application Publication
(10) Pub. No.: US 2006/0009738 A1
(43) Pub. Date: Jan. 12, 2006

(54) Method and apparatus for providing access to fluid transmitting vessels of a patient

(76) Inventor: Richard T. Rockwell JR., Pflugerville, TX (US)

Correspondence Address: E.E. "Jack" Richards, II;Winstead Sechrest & Minick P.C., P.O. Box 50784, Dallas, TX, 75201, US

(21) Appl. No.: 10/888,929

(22) Filed: Jul. 8, 2004

(51) Int. Cl. A61M 5/178

(52) U.S. Cl. 604/164.07

(57) ABSTRACT

An method and apparatus for providing access to fluid transmitting vessels of a patient, the apparatus comprising a catheter that is coupled to a hub. A needle, coupled to a needle housing, may be received within the hub and catheter. A cap is coupled to the hub and may be actuated between an open position and a closed position. In the closed position, the cap interfaces the hub thereby sealing an end of the hub. In the open position, the cap is situated so that it does not substantially seal the proximal end of the hub.

### BACKGROUND INFORMATION

1. Technical Field

The present invention relates to apparatuses and methods for providing access to fluid transmitting vessels of a patient.

2. Description of the Related Art

Health care providers routinely inject medicines into patients using typical syringes. However, patients sometimes need medicine to be administered over an extended period of time. Patients may also require many different injections of medicine over a short period of time. In such a case, administering many different “shots” to the patient would be uncomfortable to the patient. Also, some medications should be administered directly into a blood vessel, such as a vein, rather than into muscle tissue as is often the case with syringe injections. To address these conditions, health care providers often utilize the intravenous (“IV”) catheter.

The IV catheter is typically administered to the patient in the following manner. First, the patient's skin is prepared with a cleansing solution to avoid infection at the injection site. Then the clinician obtains an IV catheter. As demonstrated in FIG. 1, a typical IV catheter comprises a catheter 100 that is coupled to a hub 110. A needle 120 is also coupled to a needle housing 130. The IV catheter is typically shipped to the clinician with the needle 120 located within the hub 110 and catheter 100. An example of this arrangement can be seen in FIG. 2.

After obtaining the IV catheter, the clinician inserts the IV catheter across the patient's skin and into, for example, a fluid transmitting vessel 290 such as a vein or artery. The clinician then verifies that the tip of the needle 220 is located within a vessel. This can be done by examining whether blood flows from the vessel 290 of the patient to the needle 220. If that is the case, the clinician may grasp the needle housing 230 and remove the needle 220 from the vessel 290. The catheter 200 and hub 210 remain in place. If the catheter 200 is located within a vessel 290, pressure in the blood stream will typically cause a fluid, such as blood, to flow from the vessel 290 of the patient through the conduit 200. This flow may be continuous if the clinician does not apply pressure to the vessel 290 or seal the proximal end of the hub 280. The clinician then may connect the hub 210 to a fluid delivery device such as, for example, tubing that leads to an IV bag. In other instances, as seen in FIG. 1, the clinician may connect the hub 110 to a cap 140. The cap 140, which may or may not have a membrane disposed within, may screw onto the proximal end of the hub 180 thereby substantially sealing the proximal end of the hub 180 and, in addition, providing substantial resistance to fluid flow from the vessel of the patient though the conduit of the catheter. After the cap 140 is attached, the hub 210 is usually taped to the patient's body so that the catheter 200 does not slide out of the vessel 290.

A problem with the traditional method of applying an IV catheter, as described above, is that the clinician must attempt to keep blood from leaking from the IV catheter while he attempts to couple the cap 140 to the hub 110. Usually the clinician uses one hand to hold pressure on the vessel 290 and to hold the hub 210 in place while the other hand is used to locate the cap and couple the cap 140 to the hub 210. In a calm situation, this multitasking is challenging. In a scenario involving a traumatized patient, the problem is compounded as a clinician struggles to apply pressure to a vessel, hold the hub in place so the catheter doesn't slide out of the vessel, and locate a cap that is free to roll around on the bed or onto the floor. Once a cap falls onto the floor or comes in contact with a non-sterile environment, the cap may not be coupled to the hub in order to limit the chances for infection.

Therefore, an improved apparatus and method for providing access to fluid transmitting vessels of a patient is needed. The invention should address the difficulties a clinician experiences in starting or managing an IV catheter. More specifically, the invention should address the problems involved in starting or managing an IV catheter that are caused by interconnections, or lack thereof, between the catheter, hub and cap.

### SUMMARY DESCRIPTION

In one embodiment of the invention, a cap is coupled to a hub. More specifically, a catheter is coupled to a hub. A needle, coupled to a needle housing, may be received substantially within the hub and catheter. The cap, which is coupled to the hub at a first location, may be actuated between an open position and a closed position. In the closed position, the cap interfaces the hub in a second position, thereby sealing an end of the hub. In the open position, the cap is situated so that is does not substantially seal the proximal end of the hub.

In an alternative embodiment of the invention, a method for providing access to fluid transmitting vessels of a patient entails obtaining the previously described embodiment of the invention, inserting the needle and catheter into a fluid transmitting vessel of a patient, removing the needle from the conduit of the hub and the conduit of the catheter; and actuating the cap into the closed position.

The foregoing has outlined rather broadly the features of the present invention in order that the detailed description of the invention that follows may be better understood. Additional features and advantages of the invention will be described hereinafter, which form the subject of the claims of the invention.

### BRIEF DESCRIPTION OF THE DRAWINGS

For a more complete understanding of the present invention, and the advantages thereof, reference is now made to the following description taken in conjunction with the accompanying drawings, in which:

FIG. 1 is a side view of an example of a prior art hub, catheter, needle, needle housing and cap.

FIG. 2 is a side view of an example of a prior art catheter and needle engaging a fluid transmitting vessel of a patient.

FIG. 3 is a side view of a hub, catheter and cap, with the cap in the open position, in an embodiment of the invention.

FIG. 4 is a side view of a hub, catheter and cap, with the cap in the closed position, in an embodiment of the invention.

FIG. 5 is a side view of a hub, catheter, cap and fluid delivery device in an embodiment of the invention.

FIG. 6 is a side view of a hub, cap and means for fastening in an embodiment of the invention.

FIGS. 7A-B are side views of a catheter, hub, cap and means for fastening in an embodiment of the invention.

FIGS. 8A-B are side views of a hub, cap and multiple means for fastening in an embodiment of the invention.

### DETAILED DESCRIPTION

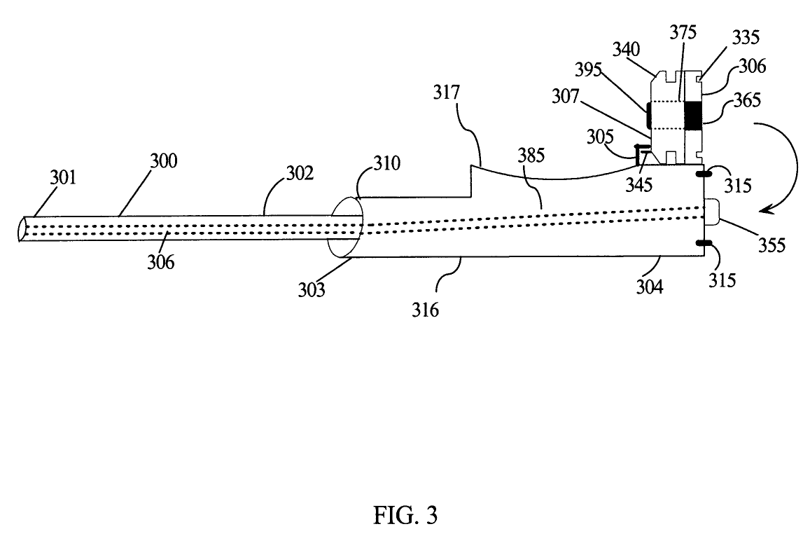

FIG. 3 illustrates an apparatus for providing access to fluid transmitting vessels of a patient. The vessels need not be limited to the transport of blood or blood parts. Suitable vessels may be closed orifices or cavities where synovial fluid, for example, resides in a somewhat stagnant state. The fluid, such as synovial fluid, may be transmitted within the closed cavity. In addition, in certain embodiments of the invention, the fluid transmitted may be in a gaseous state such as, for example, gaseous carbon dioxide or various gases present, for example, within the peritoneal cavity.

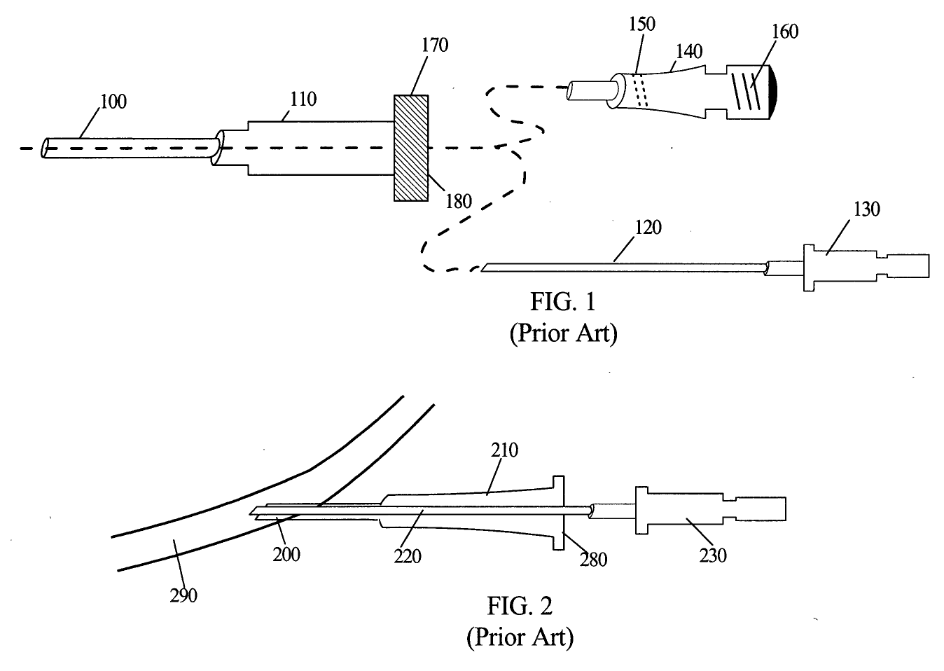

The apparatus comprises a catheter 300, sometimes known as a cannula, having a proximal end 302 and a distal end 301, and a conduit 306 extending between the proximal end of the catheter 302 and the distal end of the catheter 301. The apparatus further comprises a hub 310, having a proximal end 304 and a distal end 303, and a conduit 385 extending between the proximal end of the hub 304 and the distal end of the hub 303, wherein the proximal end of the catheter 302 is coupled to the distal end of the hub 303 and further wherein, the conduit of the hub 385 is substantially aligned with the conduit of the catheter 306. The hub 310 and catheter 300 may be contiguous with one another or may be independent parts that are coupled to one another. A needle, such as the needle 120 shown in FIG. 1, may be received substantially within the conduit of the hub 385 and the conduit of the catheter 306. In some embodiments of the invention, the needle is not necessarily received completely within the conduit of the hub or catheter. A needle housing, such as the needle housing 130 shown in FIG. 1, may be coupled to the needle. The apparatus further comprises a cap 340 coupled to the hub 310. The cap 340 may comprise a proximal end 307, a distal end 306 and a conduit 375 extending between the distal end of the cap 306 and the proximal end of the cap 307. The conduits of the cap 375, catheter 306 and hub 385 may not necessarily extend to the outer most limits of the cap 340, hub 310 and catheter 300.

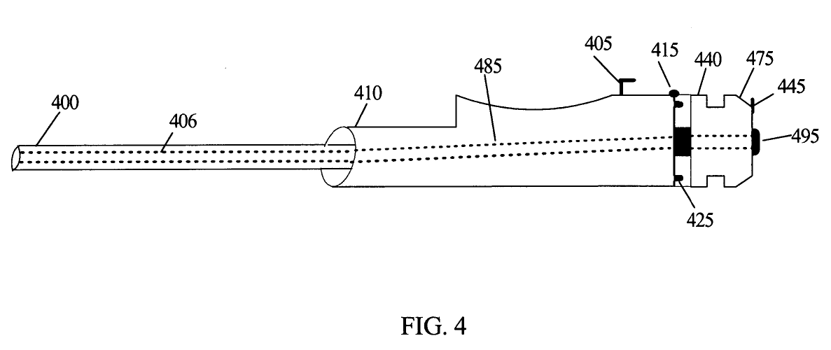

FIG. 3 depicts the cap 340 in an open position wherein the cap does not seal the proximal end of the hub 304. As a consequence, fluid may continuously travel through the catheter conduit 306 and through the hub conduit 385, moving from the distal end of the catheter 301 towards the hub 310 or from the hub 310 towards the distal end of the catheter 301. The cap 340 may be actuated from the open position to the closed position illustrated in FIG. 4. In the closed position the cap 440 interfaces the hub 410 thereby sealing the proximal end of the hub 304 and providing for the conduit of the cap 475 to be being substantially aligned with the conduit of the hub 485. In the closed position, the cap 440 disallows fluid flow to or from the vessel of a patient through the conduit of the catheter 406 and the conduit of the hub 485. The actuation of the cap 340 is not limited to any one range. For example, in an embodiment of the invention, the cap is actuated through a 90 degree range of motion as it travels from the closed position to open position. In another embodiment, the cap is actuated through a 180 degree range of motion as it travels from the closed position to the open position.

FIG. 3 illustrates a hinge 315 as an example of a means for fastening the cap 340 to the hub 310. The cap 340 may pivot about the hinge 315 when being actuated between the open and closed positions. The groove 335 and notch 325 cooperate together to fasten the cap 340 to the hub 310 in the closed position. The lever 345 and latch 305 cooperate together to fasten the cap 340 to the hub 310 in an open position. In one embodiment of the invention, a projection 355 may cooperate with orifice 365, in a closed position, to operably couple the conduit of the hub 385 to the conduit of the cap 375. The cap may comprise a membrane 395 that may be pierced by, for example, a needle thus providing the needle access to the cap conduit 375 while the cap 340 is in a closed position. The needle may comprise a traditional sharp tip or a slip tip, which is less sharp than a traditional sharp tip. Fluid may then be injected into or withdrawn from the cap conduit 375 and/or hub conduit 385. The projection 355 may cooperate with the orifice 365 in such a way that a space exists between the edges of the cap 340 and hub 310 when the cap 340 is in the closed position. The space may prevent pinching the patient's skin when actuating the cap 340 from the open position to the closed position. Also illustrated are a curved recess 306 that provides for a clinician's finger when the clinician grips the hub 310. Also, the hub 310 may have a flat surface 316 to provide stability to the hub once the catheter 300 is placed within a vessel.

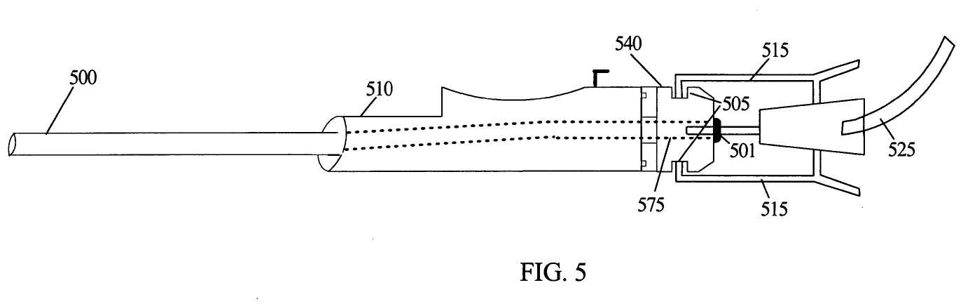

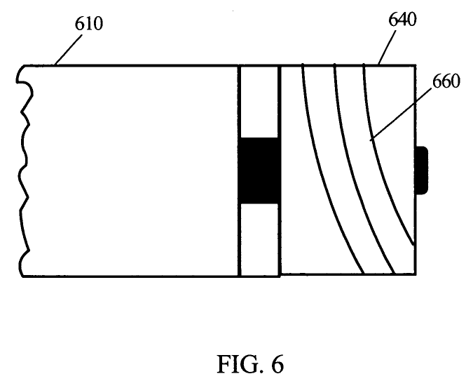

In FIG. 5, an embodiment of the invention is illustrated wherein the cap 540 is in a closed position. The cap 540 is operatively coupled to a fluid deliver device 525 such as tubing that leads to an IV bag. The fluid deliver device 525 may have projections 515 that fasten to the cap 540 by extending into recesses 505 within the cap 540. These recesses 505, in another embodiment of the invention, may exist within the hub 510. Fluid may flow from the fluid deliver device 525, across membrane 501 and into the conduit of the cap 575. FIG. 6 depicts an embodiment of the invention wherein the cap 640 has threads 660 as a means for fastening the cap 640 to a device that has reciprocating threads. Such a device may be a fluid delivery device such as tubing, a syringe or the fluid delivery device 525 of FIG. 5.

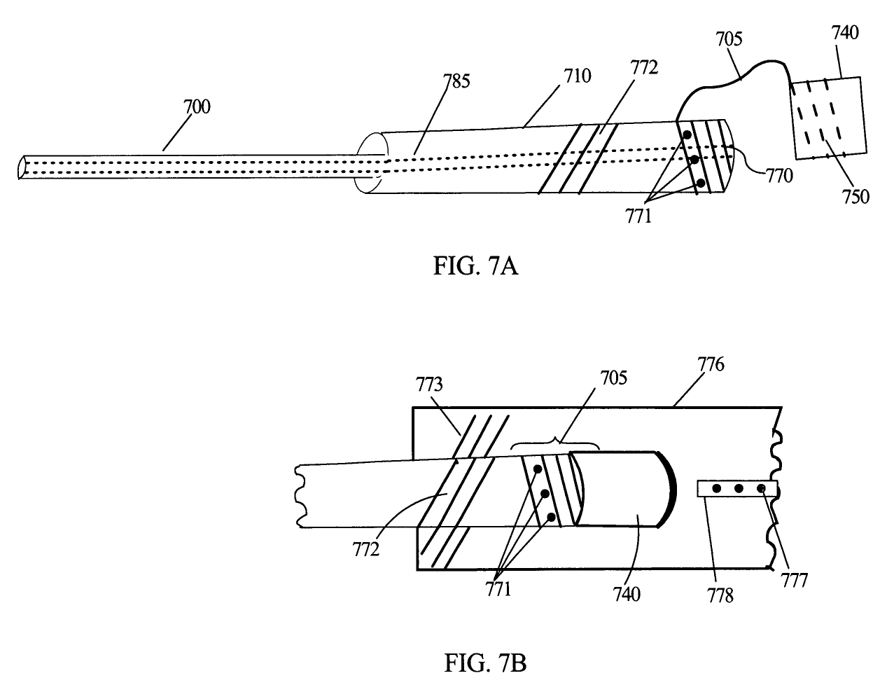

FIG. 7A depicts an embodiment of the invention wherein the cap 740 has threads 750 as an example of a means for fastening the cap 740 to a hub 710 with reciprocating threads 770. In the open position, the cap 740 remains coupled to the hub 710 due to a fastening means such as a tether 705. In the closed position, the cap 740 is coupled to the hub 710 due to a fastening means comprising reciprocating threads 770, 750 as well as the tether 705. In addition, as a person of ordinary skill in the art will further appreciate, the cap 740 may still engage the hub 710, in any number of ways, while in the open position. For example, the cap 740 may be unscrewed to the extent that holes 771 are exposed. While the holes 771 may exposed, the threads 750 of the cap 740 may still be engaged, in a limited fashion, with the threads of the hub 770. Consequently, fluid may fluid from the hub conduit 785 through the holes 771. FIG. 7B depicts a fluid transmitting device 776 that is coupled, using threads of the fluid transmitting device 773 and threads of the hub 772, to the hub 740. The hub 740 is in an open position because the cap 740 does not cover holes 771. Fluid is free to travel through the holes 771 and into the holes 777 and fluid transmitting conduit 778 of the fluid transmitting device 776.

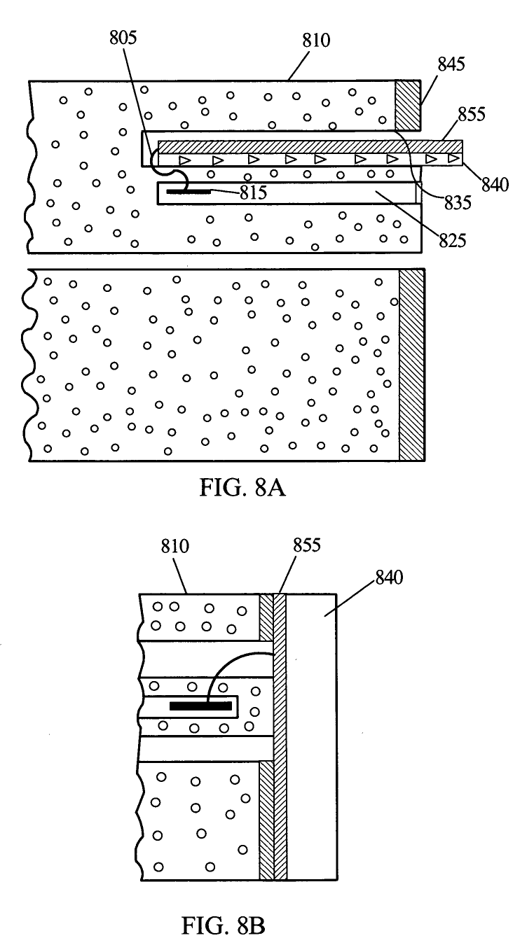

FIG. 8A demonstrates an embodiment of the invention wherein the cap 840 may reside within a recess 835 in the hub 810 when the cap 840 is in the open position. In the open position, the cap 840 is coupled to the hub 810 using a fastener such as a tether 805 that cooperates with a car 815 that travels within a chamber 825. The cap 840 may then be withdrawn from the recess 835 and then, in the closed position, coupled to the hub 810 using fastening means such as threads 855 that cooperate with reciprocal threads 845 on the hub 810.

As a person of ordinary skill in the art will appreciated, the aforementioned means for fastening one element, such as the cap 340, to another element, such as the hub 310, need not be limited to those embodiments specifically described within this application. For example, a bolt, cable, clamp, dowel, hook, key, latch, lug, nut, pin rivet or seam comprise a non-exhaustive list of fasteners that may be incorporated within various embodiments of the invention.

It will be understood that certain of the above-described structures, functions and operations of the above-described embodiments are not necessary to practice the present invention and are included in the description simply for completeness of an example embodiment or embodiments. For example, the patient need not be a human as the invention is applicable in the veterinary sector as well as human medicine sector. In addition, it will be understood that specific structures, functions and operations set forth in the above-referenced patents and publications can be practiced in conjunction with the present invention, but they are not essential to its practice. It is therefore to be understood that within the scope of the claims, the invention may be practiced otherwise than as specifically described without actually departing from the spirit and scope of the present invention. Finally, all patents, publications and standards referenced herein are hereby incorporated by reference.

What is claimed is:

1. An apparatus for providing access to fluid transmitting vessels of a patient comprising:
a catheter, comprising a proximal end and a distal end, and a conduit extending between the proximal end of the catheter and the distal end of the catheter;
a hub, comprising a proximal end and a distal end, and a conduit extending between the proximal end of the hub and the distal end of the hub, wherein the proximal end of the catheter is coupled to the distal end of the hub, and further wherein the conduit of the hub is substantially aligned with the conduit of the catheter;
a needle, comprising a proximal end and a distal end, wherein the needle may be received
substantially within the conduit of the hub and the conduit of the catheter;
a needle housing, comprising a proximal end and a distal end, wherein the proximal end of the needle is coupled to the distal end of the needle housing; and
a cap coupled to the hub at a first location, wherein the cap comprises:
a proximal end;
a distal end; and
a conduit extending between the distal end of the cap and the proximal end of the cap;
wherein, in a closed position, the cap interfaces the hub in a second location thereby substantially sealing the proximal end of the hub and providing for the conduit of the cap to be substantially aligned with the conduit of the hub; and further wherein, in an open position, the cap does not substantially seal the proximal end of the hub; the cap being capable of actuation between the open position and the closed position.

2. The apparatus of claim 1 wherein, in the open position, the cap resides substantially within the hub.

3. The apparatus of claim 1 wherein the cap is coupled to the hub at the first location using a means for fastening.

4. The apparatus of claim 3 wherein the means for fastening comprises a tether.

5. The apparatus of claim 1 wherein the cap is coupled to the hub at the second location using a means for fastening.

6. The apparatus of claim 5 wherein the means for fastening comprises a latch.

7. The apparatus of claim 1 wherein the cap further comprises a membrane.

8. The apparatus of claim 1 wherein the cap is operatively coupled to a fluid delivery device.

9. The apparatus of claim 1 wherein the hub and catheter are substantially contiguous with one another.

10. An apparatus for facilitating access to fluid transmitting vessels of a patient comprising:
a catheter, comprising a proximal region and a distal region, and a conduit extending substantially between the proximal region of the catheter and the distal region of the catheter;
a hub, comprising a proximal region and a distal region, and a conduit extending substantially between the proximal region of the hub and the distal region of the hub, the catheter being operably coupled to the hub;
a cap operably coupled to the hub at a first location;
wherein, in an open position, the cap is situated so that the cap allows continuous fluid flow from a vessel of the patient through the conduit of the catheter, and in a closed position, the cap is situated so that it is operably coupled to the hub in a second location thereby disallowing continuous fluid flow from the vessel of the patient through the conduit of the catheter, and further wherein the cap may be actuated between the open position and the closed position

11. The apparatus of claim 10 wherein, in the open position, the cap resides substantially within the hub.

12. The apparatus of claim 10 wherein the cap is operably coupled to the hub at the first location using a means for fastening.

13. The apparatus of claim 12 wherein the means for fastening comprises a tether.

14. The apparatus of claim 10 wherein the cap is operably coupled to the hub at the second location using a means for fastening.

15. The apparatus of claim 14 wherein the means for fastening comprises a latch.

16. The apparatus of claim 10 wherein the cap further comprises a membrane.

17. The apparatus of claim 10 wherein the cap is operably coupled to a fluid delivery device.

18. The apparatus of claim 10 wherein the hub and catheter are substantially contiguous with one another.

19. A method for facilitating access to fluid transmitting vessels of a patient comprising:
obtaining a device wherein the device comprises:
a catheter, comprising a proximal end and a distal end, and a conduit extending between the proximal end of the catheter and the distal end of the catheter;
a hub, comprising a proximal end and a distal end, and a conduit extending between the proximal end of the hub and the distal end of the hub, wherein the proximal end of the catheter is coupled to the distal end of the hub, and further wherein the conduit of the hub is substantially aligned with the conduit of the catheter;
a needle, comprising a proximal end and a distal end, wherein the needle may be received substantially within the conduit of the hub and the conduit of the catheter;
a needle housing, comprising a proximal end and a distal end, wherein the proximal end of the needle is coupled to the distal end of the needle housing; and
a cap coupled to the hub at a first location, the cap comprising:
a proximal end;
a distal end; and
a conduit extending between the distal end of the cap and the proximal end of the cap;
wherein, in a closed position, the cap interfaces the hub in a second location thereby substantially sealing the proximal end of the hub and providing for the conduit of the cap to be being substantially aligned with the conduit of the hub; and further wherein, in an open position, the cap does not substantially seal the proximal end of the hub; the cap being capable of actuation between the open position and the closed position inserting the needle and catheter into a fluid transmitting vessel of a patient;
removing the needle from the conduit of the hub and the conduit of the catheter; and
actuating the cap into the closed position.

20. The method of claim 19 wherein the patient is a human.
